(19) United States
(12) Patent Application Publication
(10) Pub. No.: US 2007/0049630 A1
(43) Pub. Date: Mar. 1, 2007

(54) Method of using ryanodine receptor antagonists to treat amyotrophic lateral sclerosis

(75) Inventors: Cun-Jian Dong, Irvine, CA (US); William A. Hare, Tustin, CA (US)

Correspondence Address: Robert J. Baran (T2-7H);ALLERGAN, INC., Legal Department, 2525 Dupont Drive, Irvine, CA, 92612, US

(73) Assignee: Allergan, Inc.

(21) Appl. No.: 11/448,555

(22) Filed: Jun. 7, 2006

Related U.S. Application Data
(60) Provisional application No. 60/711,179, filed on Aug. 24, 2005.

(51) Int. Cl. A61K 31/13; A61K 31/4178

(52) U.S. Cl. 514/389; 514/662

(57) ABSTRACT

The present invention provides a method of providing neural protection in in human patients suffering from amyotrophic lateral sclerosis comprising administering to said patients suffering from said amyotrophic lateral sclerosis an effective amount of a compound that is a ryanodine receptor antagonist in pharmaceutically acceptable vehicle to inhibit or prevent neuronal njury or death.

### CROSS REFERENCE TO RELATED APPLICATIONS

This application is based on, and claims the benefit of, U.S. Provisional Application No. 60/711,179, filed Aug. 24, 2005, and which is incorporated herein by reference.

### BACKGROUND OF THE INVENTION

1. Field of the Invention

The present invention concerns a method of treatment of patients suffering from amyotrophic lateral sclerosis comprising the administration of an effective amount of ryanodine receptor antagonist or negative modulator that reduces ryanodine receptor activity.

2. Description of the Related Art

There is compelling evidence that abnormally elevated intracellular free calcium is one of the early events in the chain of reactions leading to neuronal damage under pathological conditions that range from acute neural injuries, such as stroke, to more chronic indications, such as Alzheimer's disease. High intracellular free calcium can cause mitochondrial injury and activate various types of enzymes, such as proteases, nitric oxide synthases and endonucleases. These calcium-induced/activated cellular responses are believed to mediate cytotoxicity that eventually leads to neuronal death.

There are two major mechanisms that can cause elevation of intracellular free calcium: 1) calcium influx from extracellular space through calcium and non-selective cation channels on the cell membrane, and 2) calcium release from intracellular stores, such as endoplasmic reticulum and mitochondria, through specialized receptor-channel complex, such as ryanodine receptor channels. These two mechanisms often interact. For example, calcium entered the cell through ion channels on the cell membrane can trigger more calcium release from intracellular stores. This calcium-induced calcium release (CICR) has been demonstrated to contribute to neuronal damage under pathological conditions.

Amyotrophic lateral sclerosis (incidence of 1.4 to 4.7/100.000) also named motor neuron disease, is a degenerative disease characterized by progressive paralysis which affects elderly subjects (65-70 years), developing into complete paralysis and death and in a short time.

As stated in U.S. Pat. No. 6,855,694 B2, several biochemical and genetic factors seem to be involved in the pathogenesis of ALS, which remains however to be still elucidated. An increase in some intracellular proteins (cytoskeleton) which affect cell activity and neurotransmission, may be the cause of amyotrophic lateral sclerosis.

According to a recent hypothesis, the first step in the onset of ALS appears to be connected with an increase of toxic factors such as oxygen radicals and the cited formation of protein cytoskeleton whereas the degenerative and progressive phase would seem to be at least partially activated and sustained by autoimmune mechanisms.

5% of the cases of ALS are of familial origin and 95% of the cases are sporadic. The physio-pathological origin of the sporadic forms of ALS remains unknown. Several hypotheses have been proposed. The motor neurons degeneration could result from an alteration in the metabolism of glutamate leading to an increase in the concentration of this exciter amino acid in the motor cortex and the spinal cord (“excitotoxic” hypothesis, review in Rothstein, 1995). The possibility of an autoinmune component has likewise been put forward on the basis of the presence of auto-antibody against the voltage-sensitive calcium channels in certain patients (review in Appel et al., 1995). The implication of environmental factors such as exposure to certain viruses (review in Gastaut, 1995), or to aluminium (Yase, 1984) is likewise possible. (See U.S. Pat. No. 6,723,315 B1.)

There is no specific treatment at present for ALS.

No experimental therapy seems particularly promising.

One of the major difficulties in developing an effective treatment for ALS is due to the lack of a reliable and predictive animal model so that the only definitive evidence on the actual effectiveness of a new therapy has to be obtained from clinical tests. However, it is believed that the teachings of the present invention may overcome this difficulty.

Thus, it is evident that there is an unmet need for agents that have neuroprotective effects that can stop or retard the progressive damage to neurons resulting from abnormally elevated intracellular free calcium caused by various noxious provocations.

Dantrolene, a skeletal muscle relaxant, has been found to be an antagonist of the ryanodine receptor-channel complex (See Biochemistry 2001, 40, 531-542). Dantrolene blocks calcium release from ryanodine channels when it binds to the receptor.

Dantrolene is 1-[[5-(p-Nitrophenyl)furfurylidene]amino]hydantoin.

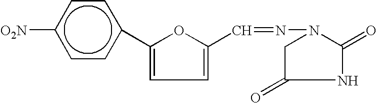

It has now been found that ALS can be effectively treated by administering to affected patients protein.

### SUMMARY OF THE INVENTION

A new method of protecting the motor neurons of a mammal from noxious provocations has been discovered. The present method uses a ryanodine receptor antagonist to prevent or ameliorate damage to neurons caused by noxious provocations that induce excessive calcium release from intracellular stores via ryanodine receptor channels. These noxious provocations, including excitotoxicity, ischemia, hypoxia, mitochondrial dysfunction, and oxidative injury, are associated with acute and chronic neural disorders. The method comprises administering to the mammal either systemically, topically, or epidurally an effective amount of one or more ryanodine receptor antagonists, such as dantrolene.

For protection of motor neurons in humans suffering from amyotrophic lateral sclerosis or ALS, the active compounds (or mixtures or salts thereof) may be administered in accordance with the present invention to the patient admixed with an acceptable carrier. Any suitable, e.g., conventional carrier may be employed. A carrier is acceptable if it has substantially no long term or permanent detrimental effect on the patient to which it is administered. Examples of acceptable carriers include physiological saline and other aqueous media. In accordance with the invention, the active compounds are preferably soluble in the carrier which is employed for their administration, so that the active compounds are administered to the patient in the form of a solution. Alternatively, a suspension of the active compound or compounds (or salts thereof) in a suitable carrier may also be employed.

In accordance with the invention the active compounds (or mixtures or salts thereof) are administered in an acceptable carrier in sufficient concentration so as to deliver an effective amount of the active compound or compounds to the patient. Preferably, the therapeutic solutions contain one or more of the active compounds in a concentration range of approximately 0.0001% to approximately 10% (weight by volume) and more preferably approximately 0.005% to approximately 0.5% (weight by volume).

Any method of administering drugs to a patient may be employed to administer, in accordance with the present invention, the active compound or compounds to the patient to be treated.

An exemplary formulation is shown below in Table 1. The abbreviation q.s. means a quantity sufficient to obtain the result or to make volume.

TABLE I

| Ingredient | Amount (% W/V) |
|---|---|
| Active Compound in accordance | about 0.0001 to about 1 |
| with the invention, | |
| Preservative | 0-0.10 |
| Vehicle | 0-40 |
| Tonicity Adjustor | 1-10 |
| Buffer | 0.01-10 |
| PH Adjustor | q.s pH 4.5-7.5 |
| Antioxidant | as needed |
| Purified Water | as needed to make 100% |

Various preservatives may be used in the preparation described in Table I above. Preferred preservatives include, but are not limited to, benzalkonium chloride, chlorobutanol, thimerosal, phenylmercuric acetate, and phenylmercuric nitrate. Likewise, various preferred vehicles may be used in such preparation. These vehicles include, but are not limited to, polyvinyl alcohol, povidone, hydroxypropyl methyl cellulose, poloxamers, carboxymethyl cellulose, hydroxyethyl cellulose, and purified water.

Various buffers and means for adjusting pH may be used so long as the resulting preparation is pharmaceutically acceptable. Accordingly, buffers include but are not limited to, acetate buffers, citrate buffers, phosphate buffers, and borate buffers. Acids or bases may be used to adjust the pH in these formulations as needed.

In a similar vein, antioxidants include, but are not limited to, sodium metabisulfite, sodium thiosulfate, acetylcysteine, butylated hydroxyanisole, and butylated hydroxytoluene.

The compositions of this invention may be administered to the patient as often as necessary to obtain the desired concentration that affords neuroprotection. The drug is administered as frequently as necessary to maintain desired concentration or range of concentrations at all times. In other words, the solution (or other formulation) which contains the ryanodine antagonist or negative modulator as the active ingredient, is administered to the patient as often as necessary to maintain the beneficial neuroprotective effect of the active ingredient in the patient. Those skilled in the art will recognize that the frequency of administration depends on the precise nature of the active ingredient and its concentration in the formulation. Within these guidelines it is contemplated that the formulation of the present invention will be administered to the patient approximately once or twice daily.

This new method is particularly effective when administered as a prophylactic treatment, i.e. before damage to the nerve cells has taken place, or before long-term progression of the disease state, has taken place. Without wishing to be held to a particular theory regarding the role that the compounds of the present invention play in neuroprotection, applicants hypothesize that the compounds and methods described inhibit the intracellular Ca+2 release.

Thus it is further contemplated that the compounds of the present invention can advantageously be used in combination with compounds that inhibit cell death. Such cell death inhibiting compounds include NMDA antagonists especially memantine.

### DETAILED DESCRIPTION OF THE INVENTION

The present invention is related to methods of using ryanodine receptor antagonists to protect neurons, particularly the motor neurons, from injuries caused by ALS.

As mentioned above, excessive release of calcium from intracellular stores under disease conditions is cytotoxic to neurons. A recent study on sporadic ALS patients has provided evidence that abnormal post-transcriptional modification of mRNA encoding a subtype of glutamate receptor (AMPA receptor) may make the receptor more Ca⁺⁺ permeable, which may promote sporadic ALS. We think that Ca⁺⁺ influx through this abnormal AMPA receptor can trigger excessive calcium release from intracellular stores, and eventually leads to the death of motor neurons.

There is strong evidence that damage to CNS neurons often has two stages: Primary and secondary degeneration. Initially, direct neuronal insults, such as local ischemia, trauma etc., lead to degeneration of the affected neurons. However, the associated pathophysiological and biochemical events occurring in the injured neurons are probably responsible for the subsequent progressive (secondary) degeneration of the neighboring neurons that are not directly affected by the primary insults. These secondary effects largely determine the long-term functional outcome.

The immediate injury-induced response strongly influences the subsequent degenerative response. Treatment that reduces or attenuates the injury to the primary insults is therefore likely to generate optimal results by preventing or delaying the secondary degenerative processes.

It is believed that neuroprotection is conferred upon motor neurons by administration of a ryanodine antagonist, e.g. dantrolene, to a patient suffering from ALS within a period prior to, or following an primary insult to the motor neurons but prior to cell death,

The terms noxious actions or noxious provocations are defined as an occurrence which is harmful or destructive to a nerve cell. It is not limited to events extrinsic to the mammal being treated but includes disease states and pathological occurrences or events, such as, for example, stroke or heart attack, that are harmful or destructive to the nerve cell via a chain of events. Non-limiting examples of noxious actions include: compressive or mechanical effects or trauma or stress factors, such as glutamate neurotoxicity or impaired blood flow to the nerves (ischemia).

Human Dosage And Administration

The methods of this invention are useful in treating humans.

According to this invention, patients are treated with pharmaceutically effective amount of a neuroprotective agent for a period of time and at a time such that noxious provocations do not kill or permanently damage the nerve cells. Protective agents may be administered orally or by any other appropriate means of delivery described below or known in the art.

In accordance with this invention, pharmaceutically effective amounts of a protective agent can be administered alone to treat neural injury or to prevent nerve cell death. Alternatively a protective agent may be administered sequentially or concurrently with another drug. The most effective mode of administration and dosage regimen of protective agent will depend on the severity and course of the disease, previous therapy, the patient's health status, and response to the drug and the judgment of the treating physician. Generally, the neuroprotective agent should be administered in a dose to achieve a serum concentration of 0.01 nM to 20 μM. Preferably the neuroprotective agent is administered prior to injury to the nerve cells, but can be administered after injury has occurred with lessened effect.

Conventional modes of administration and standard dosage regimens of neuroprotective agents can be used. Optimal dosages for coadministration of a drug, with a neuroprotective agent can be determined using methods known in the art. Dosages of neuroprotective agents may be adjusted to the individual patient based on the dosage of the drug with which the agent is coadministered and the response of the patient to the treatment regimen. The neuroprotective agent may be administered to the patient at one time or over a series of treatments.

The agent may be administered locally, or by intrathecal or epidural administration for spinal protection. Many of the agents of the invention can be administered systemically, e.g., orally, or intravenously, or by intramuscular injection.

The composition used in these therapies may also be in a variety of forms. These include, for example, solid, semi-solid, and liquid dosage forms, such as tablets, pills, powders, preserved or non-preserved liquid solution or suspension, liposomes, suppositories, injectable and infusible solutions. The compositions also preferably include conventional pharmaceutically acceptable carriers which are known to those of skill in the art.

The following non-limiting examples describe assays and measurements used in 1) evaluating efficacy of neuroprotecting agents and 2) selecting ryanodine antagonists other than dantrolene.

### EXAMPLE 1

Assay for Selecting Ryanodine Antagonists Other than Dantrolene.

Assays for determining ryanodine antagonist may be conducted following procedures modified from that described by Laver et al., (J. Physiol. 537:763-778, 2001). Briefly, purified ryanodine receptor-channel complexes are incorporated into planar phospholipid bilayers with resting calcium gradient similar to that in a normal neuron at rest (100 nM cytoplasmic and 1 mM luminal). The level of channel activation can be determined in the presence of various ligands that activate ryanodine receptors. Effective antagonistic action of the compounds to be selected can be determined by a reduction of agonist-induced activation of the channel. The specificity of the antagonists can be determined by commercially available standard screens, such as NovaScreens.

See also, U.S. patent application Ser. No. 11/362,319 which was filed on Feb. 23, 2006 in the names of the present inventors for additional methods of determining ryanodine antagonists for use in the method of the present invention.

While this invention has been described with respect to various specific examples and embodiments, it is to be understood that the invention is not limited thereby and should only be construed by interpretation of the scope of the appended claims.

What is claimed is:

1. A method of providing neural protection in in human patients suffering from amyotrophic lateral sclerosis comprising administering to said patients suffering from amyotrophic lateral sclerosis an effective amount of a compound that is a ryanodine receptor antagonist or negative modulator that reduces ryanodine receptor activity in pharmaceutically acceptable vehicle to inhibit or prevent nerve cell injury or death.

2. The method of claim 1 wherein said disorder is a result of glutamate induced excitotoxic effects on nerve cells.

3. The method of claim 1 wherein said compound is administered in combination with a NMDA antagonist.

4. The method of claim 3 wherein said NMDA antagonist is memantine.

5. The method of claim 1 wherein said compound is dantrolene.

6. The method of claim 1 wherein said compound is administered in an amount sufficient to achieve a serum concentration of from 0.01 nM to 20 μM.

7. The method of claim 1 wherein said compound is administered orally, or by intravenous injection.
